(19) United States
(12) Patent Application Publication
(10) Pub. No.: US 2009/0221532 A1
(43) Pub. Date: Sep. 3, 2009

(54) Methods Of Dosing Propofol Prodrugs For Inducing Mild To Moderate Levels Of Sedation

(75) Inventors: Ekaterina Gibiansky, North Potomac, MD (US); Leonid Gibiansky, North Potomac, MD (US)

Correspondence Address: BANNER & WITCOFF, LTD., 1100 13th STREET, N.W., SUITE 1200, WASHINGTON, DC, 20005-4051, US

(73) Assignee: MGI GP, INC., Baltimore, MD (US)

(21) Appl. No.: 11/995,127

(22) PCT Filed: Jul. 11, 2006
(86) PCT No.: PCT/US06/26840

§ 371 Date: Aug. 1, 2008

Related U.S. Application Data
(60) Provisional application No. 60/698,404, filed on Jul. 12, 2005.

(51) Int. Cl. A61K 31/661

(52) U.S. Cl. 514/130

(57) ABSTRACT

A dosage of a propofol prodrug needed for inducing mild to moderate sedation levels in a patient is calculated based on a patient's lean body mass. It has been discovered dosages based on gross body mass may result in overdosing, particularly for obese patients. In another aspect, a dosage suitable for inducing mild to moderate sedation levels in a patient who is at least 60 years of age is determined. A weight-appropriate dosage for the patient is determined and then adjusted by an age-based factor. For example, the dosage needed to produce a sedated state or other effect in a patient who is 60 years of age or older may be about 0.6-0.8 times the dosage needed to produce a corresponding effect in a younger patient of the same weight.

This application claims priority to provisional U.S. Application Ser. No. 60/698,404, filed Jul. 12, 2005, herein incorporated by reference.

### FIELD OF THE INVENTION

### Background of the Invention

The outpatient setting has become increasingly popular for various types of medical procedures requiring sedation. In outpatient colonoscopy, for example, benzodiazepines are widely used for sedation. The combination of midazolam HCl with a narcotic analgesic is a very common drug regimen for providing mild to moderate sedation and analgesia. Gastroenterologists have searched for alternative treatments to use in the outpatient setting that would provide a faster recovery time and accelerated “street-fitness” after patient sedation for outpatient surgical and diagnostic procedures.

The use of injectable anesthetic agents generally, and of propofol specifically, in the induction and maintenance of general anesthesia has gained widespread acceptance in anesthetic care over the last 15 years. Intravenous anesthesia with propofol has been described to have several advantages over preexisting methods, such as more readily tolerated induction, since patients need have no fear of masks, suffocation, or the overpowering smell of volatile anesthetics; rapid and predictable recovery; readily adjustable depth of anesthesia by adjusting the IV dose of propofol; a lower incidence of adverse reactions as compared to inhalation anesthetics; and decreased dysphoria, nausea, and vomiting upon recovery from anesthesia [Padfield N L, Introduction, history and development. In: Padfield N L (Ed.) Ed., Total Intravenous Anesthesia. Butterworth Heinemann, Oxford 2000].

In addition to its sedative and anesthetic effects, propofol has a range of other biological and medical applications. For example, it has been reported to be an anti-emetic [McCollum J S C et al., Anesthesia 43 (1988) 239], an anti-epileptic [Chilvers C R, Laurie P S, Anesthesia 45 (1990) 995], and an anti-pruritic [Borgeat et al., Anesthesiology 76 (1992) 510]. Anti-emetic and anti-pruritic effects are typically observed at subhypnotic doses, i.e., at doses that achieve propofol plasma concentrations lower than those required for sedation or anesthesia. Antiepileptic activity, on the other hand, is observed over a wider range of plasma concentrations [Borgeat et al., Anesthesiology 80 (1994) 642]. Short-term intravenous administration of subanesthetic doses of propofol has also been reported to be remarkably effective in the treatment of intractable migraine and nonmigrainous headache [Krusz J C, et al., Headache, 40 (2000) 224-230]. It has further been speculated that propofol may be useful as an anxiolytic [Kurt et al., Pol. J. Pharmacol. 55 (2003) 973-7], neuroprotectant [Velly et al., Anesthesiology 99 (2003) 368-75], muscle relaxant [O'Shea et al., J. Neurosci. 24 (2004) 2322-7] and, due to its antioxidant properties in biological systems, may further be useful in the treatment of inflammatory conditions, especially inflammatory conditions with a respiratory component, and in the treatment of neuronal damage related to neurodegeneration or trauma. Such conditions are believed to be associated with the generation of reactive oxygen species and therefore amenable to treatment with antioxidants. See, e.g., U.S. Pat. No. 6,254,853 to Hendler et al.

Propofol typically is formulated for clinical use as a oil-in-water emulsion. The formulation has a limited shelf-life and has been shown to be sensitive to bacterial or fungal contamination, which has led to instances of postsurgical infections [Bennett S N et al., N Engl J Med 333 (1995) 147]. Due to the dense, white color of the formulation, bacterial or fungal contamination cannot be detected by visual inspection of the vial in the first instance.

Not only is propofol poorly water soluble, but it also causes pain at the injection site, which must often be alleviated by using a local anesthetic [Dolin S J, Drugs and pharmacology. In: N. Padfield, Ed., Total Intravenous Anesthesia. Butterworth Heinemann, Oxford 2000]. Due to its formulation in a lipid emulsion, its intravenous administration is also associated with undesirable hypertriglyceridemia in patients, especially in patients receiving prolonged infusions [Fulton B and Sorkin E M, Drugs 50 (1995) 636]. Its formulation as a lipid emulsion further makes it difficult to co-administer other IV drugs. Any physical changes to the formulation, such as a change in lipid droplet size, can lead to changes in the pharmacological properties of the drug and cause side effects, such as lung embolisms.

It has further been reported that the use of propofol in anesthesia induction is associated with a significant incidence of apnea, which appears to be dependent on dose, rate of injection, and premedication [Reves, J G, Glass, P S A, Lubarsky D A, Nonbarbiturate intravenous anesthetics. In: R. D. Miller et al., Eds, Anesthesia. 5^{th} Ed. Churchill Livingstone, Philadelphia, 2000]. Respiratory consequences of administering anesthetic induction doses of propofol, including a reduction in tidal volume and apnea, occur in up to 83% of patients [Bryson et al., Drugs 50 (1995) at 520]. Induction doses of propofol are also known to have a marked hypotensive effect, which is dose- and plasma concentration-dependent [Reves et al., supra]. The hypotension associated with peak plasma levels after rapid bolus injection of propofol sometimes requires the use of controlled infusion pumps or the breaking-up of the induction bolus dose into several smaller incremental doses. Further, the short duration of unconsciousness caused by bolus induction doses renders propofol suitable for only brief medical procedures. For all the above reasons, propofol for induction and/or maintenance of anesthesia must normally be administered under the supervision of an anesthesiologist or other staff qualified in airway maintenance, and is often considered inappropriate for use by non-anesthesiologists in an ambulatory or day case setting.

In addition to its use in induction and maintenance of anesthesia, propofol has been used successfully as a sedative to accompany either local or regional anesthesia in conscious patients. Its sedative properties have also been exploited in diagnostic procedures that have an unsettling effect on conscious patients, such as colonoscopy or imaging procedures. Propofol has also been used as a sedative in children undergoing diagnostic imaging procedures or radiotherapy. A recent development is that of patient-controlled sedation with propofol. This technique is preferred by patients and is as effective as anesthesiologist-administered sedation.

Compared with the widely used sedative midazolam or other such agents, propofol provided similar or better sedative effects when the quality of sedation and/or the amount of time that patients were at adequate levels of sedation were measured [see Fulton B and Sorkin E M, Drugs 50 (1995) 636]. The faster recovery and similar or less amnesia associated with propofol makes it an attractive alternative to other sedatives, particularly for patients requiring only short sedation. However, because of the potential for hyperlipidemia associated with the current propofol formulation, and the development of tolerance to its sedative effects, the usefulness of propofol for patients requiring longer sedation is less well established.

Due to its very low oral bioavailability, propofol in its commercially available formulations is generally recognized as not suitable for other than parenteral administration, and generally must be injected or infused intravenously. While propofol is administered intravenously in a clinical setting, it has been suggested that it could be delivered for certain indications via other non-oral routes, such as via inhalation using a nebulizer, transmucosally through the epithelia of the upper alimentary tract, or rectally in the form of a suppository [see, e.g. Cozanitis, D. A., et al., Acta Anaesthesiol. Scand. 35 (1991) 575-7; see also U.S. Pat. Nos. 5,496,537 and 5,288,597]. However, the poor bioavailability of propofol when administered by any other than the intravenous route has hampered the development of such treatments.

The development of water soluble and stable prodrugs of propofol, which is described in U.S. Pat. No. 6,204,257 to Stella et al., has made it possible to address these heretofore unmet needs, and to explore the pharmaceutical advantages of an aqueous propofol-prodrug as a therapeutic agent. The prodrugs differ from propofol in that the 1-hydroxy-group of propofol is replaced with a phosphonooxymethyl ether group:

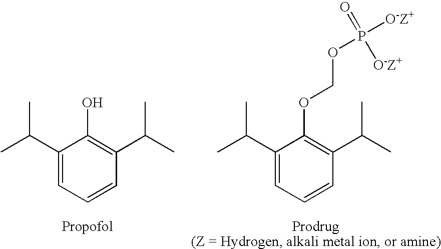

While the present invention is not bound by any theory, the prodrug is believed to undergo hydrolysis by alkaline phosphatases to release propofol.

Stella reports that the prodrug has good stability at pH levels suitable for making pharmaceutical formulations, and quickly breaks down in vivo under physiological conditions when administered intravenously. The prodrugs possess a favorable pharmacological profile as therapeutics for sedation and anesthetic care, and for the treatment of conditions such as migraine, epilepsy, pruritus, anxiety, insomnia, nausea, and other medical conditions.

### BRIEF SUMMARY OF THE INVENTION

In one aspect, the present invention is directed to a method of determining a dosage of a compound of Formula I effective for inducing mild to moderate sedation levels in a patient:

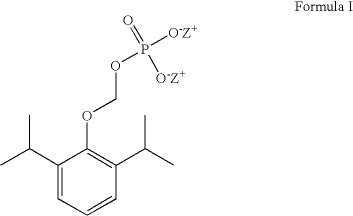

wherein each Z is independently selected from the group consisting of hydrogen, alkali metal ion, and amine. The method comprises determining a patient's lean body mass and selecting a dosage based on lean body mass. It has been discovered that lean body mass, rather than gross body mass, is an advantageous parameter for determining prodrug dosages for weight-proportional dosing of subjects requiring sedation for short surgical or diagnostic procedures, such as colonoscopies. This finding is expected to have significant therapeutic implications particularly for the dosing of overweight or obese subjects.

In another aspect of the invention, a method is provided for determining a dosage of a compound of Formula I suitable for inducing mild to moderate sedation levels in a patient who is at least 60 years of age. The method comprises determining a weight-appropriate dosage for the patient and then adjusting the weight-appropriate dosage by an age-based factor. For example, the dosage needed to induce mild to moderate sedation levels in a patient who is 60 years of age or older may be about 0.6 to about 0.8 times the dosage needed to produce a corresponding effect in a younger patient of the same weight.

### BRIEF DESCRIPTION OF THE DRAWINGS

FIGS. 1A and 1B show plasma concentrations as a function of time following the initial dose of O-phosphonooxymethyl propofol disodium salt (FIG. 1A) and propofol (FIG. 1B).

FIG. 2 shows the mean Modified OAA/S over time at each procedural period by initial bolus dose for Example 1A;

FIG. 3 is a graph showing the mean Modified OAA/S score over time at each procedural period by initial bolus dose for Example 1B;

FIGS. 4A and 4B illustrate the percent of time that the patients were at each Modified OAA/S score during colonoscopy by initial bolus dose for Examples 1A and 1B, respectively;

FIG. 5 illustrates the expected Modified OAA/S score versus effect-compartment concentration for probabilistic (solid) and continuous (dash) models; and

FIG. 6 illustrates the correlation between the expected and observed MOAA/S scores.

### DETAILED DESCRIPTION OF THE INVENTION

The active agent for inducing sedation is a water-soluble compound of Formula I:

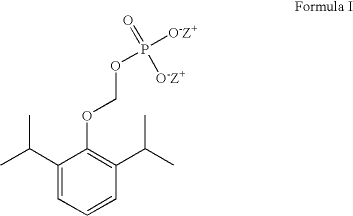

or a pharmaceutically acceptable salt thereof, wherein each Z is independently selected from the group consisting of hydrogen, alkali metal ion, and amine. Each Z preferably is an alkali metal ion, especially a sodium ion such that the prodrug is O-phosphonooxymethyl propofol disodium salt.

Methods for the chemical synthesis of the compounds of Formula I are described in U.S. Pat. No. 6,204,257 to Stella et al. and in WO 03/059255 A2, each of which is incorporated by reference herein. The propofol prodrug of Formula I is water soluble and can be formulated in aqueous solutions or in other suitable pharmaceutical compositions.

The compounds of Formula I can be readily formulated for administration to a patient by combining them with well-known pharmaceutically acceptable carriers. Such carriers enable the compounds of the invention to be formulated as tablets, pills, capsules, liquids, quick-dissolving preparations, gels, syrups, slurries, suspensions and the like, for oral ingestion by a patient to be treated. Pharmaceutical preparations for oral use can be obtained by mixing the compound with a solid excipient, optionally grinding a resulting mixture, and processing the mixture of granules, after adding suitable auxiliaries, if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In general, the pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. If desired, disintegrating agents may be added, such as the cross-linked polyvinyl pyrrolidone, agar, or alginic acid or a salt thereof such as sodium alginate or a number of others disintegrants (see, for example, Remington's Pharmaceutical Sciences, Mack Publishing Co., Easton, Pa., Remington's Pharmaceutical Sciences, Mack Publishing, Easton, Pa., 20th Ed, 2000). For liquid formulations, any pharmaceutically acceptable aqueous medium may be used, such as sterile water, physiological saline, or a mixture of water and an organic solvent, such as propylene glycol, ethanol, and the like. The concentration of the compound of Formula I in the formulation most often ranges from about 0.5 to about 35% (w/v), more usually from about 1 to about 20%. For example, O-phosphonooxymethyl propofol disodium salt can be formulated as a sterile solution at concentrations of 20 and 35 mg/mL suitable for i.v. administration. International patent application publication WO 2003/057153 describes aqueous formulations of the prodrug of this invention suitable for parenteral, particularly intravenous, administration.

As will be appreciated by those skilled in the art, many factors influence the choice of appropriate dosage, mode, and schedule of administration. For example, the appropriate dosage for inducing mild to moderate levels of sedation in a patient may depend on whether the patient is a human, or another mammal, or is a non-mammalian patient; it may depend on the patient's age, weight, sex, diet, health, underlying medical condition, and the like. Therefore, an anesthesiologist, veterinarian, or other medical, science, or health practitioner skilled in the art will be able to devise, in light of the guidance provided herein, and without undue experimentation, an appropriate treatment protocol.

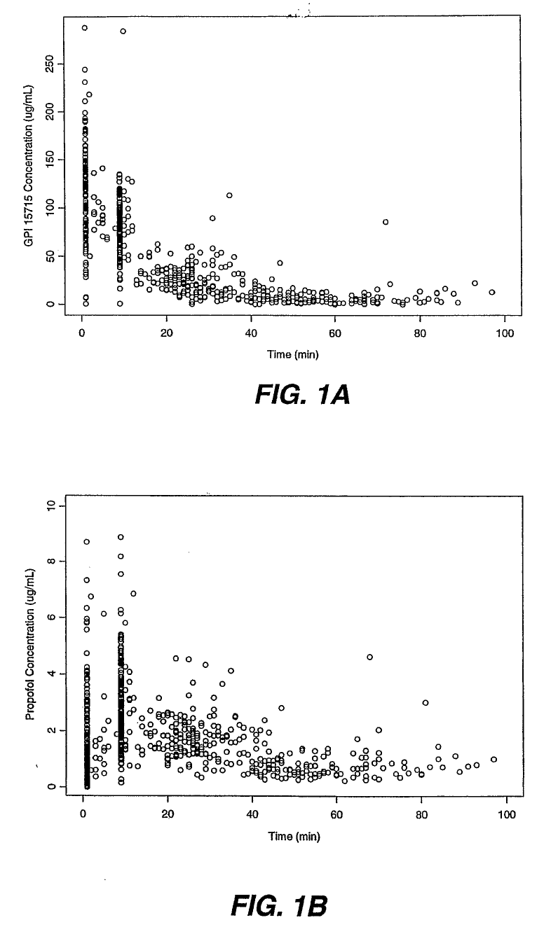

FIGS. 1A and 1B show plasma concentrations as a function of time following initial administration. Plasma samples were analyzed by MDS Pharma Services, St. Laurent, Quebec, Canada H4R 2N6. A validated high performance liquid chromatography (HPLC) assay with fluorescence detection with a lower limit of quantitation (LOQ) of 5 ng/mL was used for the analysis of propofol concentrations. A validated HPLC mass spectrometric method with an LOQ of 5 ng/mL was used for the analysis of O-phosphonooxymethyl propofol disodium salt concentrations.

A pretreatment agent, such as fentanyl citrate injection (sometimes referred to herein as “fentanyl”) preferably is administered to mitigate paresthesias, e.g., a transient sensation of burning, heat, or tingling that occurs soon after the onset of infusion and most commonly in the anal and genital region. The origin of paresthesias is believed to be a manifestation of the prodrug, as other phosphonoxy prodrugs are known to manifest this sensation.

The term “lean body mass,” as used herein, refers to a weight of lean body and is obtained by subtracting body fat weight from gross body weight. For the purposes of the present invention, lean body mass was calculated according to the formula of Hallynck et al. (Hallynck, T H, Soep H H, et. al., Br. J. Clin. Pharmacol. 11, 1981, 523-526). Briefly, for male subjects, lean body weight (LBW) is calculated according to the following formula: LBW_{[kg], males}=1.10*weight_{[kg]}−128*(weight_{[kg]/height}_{[cm])}². For female subjects, the following formula is applied: LBW_{[kg], females}=1.07*weight_{[kg]}−148*(weight_{[kg]}/height_{[cm]})².

In one embodiment of the present invention, a conscious sedated state is induced, or maintained over an extended period of time, in a patient by parenteral administration of an effective amount of the propofol prodrug of Formula I. In this embodiment, the effective amount is determined by calculating the patient's lean body weight and then selecting a weight-proportional dose based on the patient's lean body weight. In a preferred aspect of this embodiment, the patient is overweight or obese.

“Overweight” and “obese” as used herein denote ranges of weight that are greater than what skilled persons consider healthy for a given height. The terms also identify ranges of weight that have been shown to increase the likelihood of certain diseases and other health problems. For adults, overweight and obese weight ranges are determined by using weight and height to calculate the “body mass index” (BMI) according to the following formula: BMI=weight_{[kg]}/height_{[meter]}². For children and adolescents up to age 20, BMI is determined according to age- and gender-specific charts [see, e.g., Hammer L D, Kraemer H C, Wilson D M, Ritter P L, Dornbusch S M. Standardized percentile curves of body-mass index for children and adolescents. American Journal of Disease of Child. 1991; 145:259-263]. As used herein, an adult with a BMI between 25 and 29.9 is considered overweight. A BMI of 30 and above is considered obese. For most adults, BMI correlates with, but does not provide a reliable measure for, body fat. As a result, some individuals, such as athletes, may have a BMI that identifies them as overweight even though they do not have excess body fat. The relation between fatness and BMI differs with age and gender. For example, women are more likely to have a higher percent of body fat than men for the same BMI. On average, older people may have more body fat than younger adults with the same BMI.

Dose ranges suitable for inducing and maintaining a conscious sedated state in a patient by administration of a prodrug of Formula I have been described previously in international patent application publication WO 03/086413. In the present invention, suitable doses applicable specifically to an overweight or obese patient are selected based on such overweight or obese patient's lean body weight rather than gross body weight. For example, a conscious sedated state can be induced or maintained in a patient with normal weight by single or repeated bolus injections of the prodrug of Formula I at a range from about 2 mg/kg to about 20 mg/kg gross body weight, preferably from about 5 mg/kg to about 15 mg/kg, and more preferably from about 5 mg/kg to about 10 mg/kg gross body weight. In an overweight or obese patient, suitable doses range from about 3 mg/kg to about 30 mg/kg lean body weight, preferably from about 7.5 mg/kg to about 23 mg/kg, and more preferably from about 7.5 mg/kg to about 15 mg/kg lean body weight.

Propofol is known to have a reduced clearance and initial volume of distribution in the elderly. Older patients require lower doses for any given effect, in many cases as little as 50% of an expected (e.g., weight-based) dose. It has now been discovered that patients 65 years of age and older tend to be more sensitive not just to propofol when administered in its native form, but also to propofol derived from intravenous administrations of the prodrug of Formula I. A strictly weight-based dosage of the prodrug of Formula I for elderly patients thus may result in a higher degree of sedation than may be needed or desired.

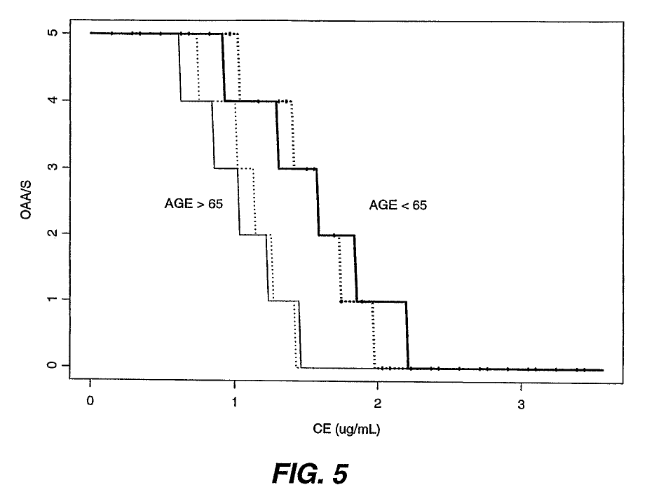

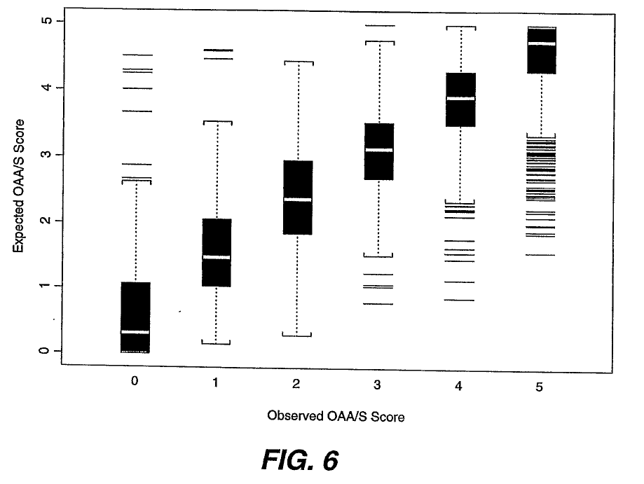

FIG. 5 illustrates the expected Modified OAA/S score versus effect-compartment concentration for probabilistic (solid) and continuous (dash) models. These models predicted that patients >65 years of age will achieve a given Modified OAA/S score at lower effect-compartment concentrations than patients <65 years of age. FIG. 6 illustrates the correlation between expected and observed MOAA/S scores. Results were similar for both the probabilistic and continuous models.

A dosage of a compound of Formula I for inducing mild to moderate sedation levels in a patient who is at least 60 years of age can be calculated by first determining a weight-appropriate dosage for the patient and then adjusting the weight-appropriate dosage by an age-based factor. Typically, the dosage needed to produce a sedated state or other effect in a patient between 60 and 85 years of age, is about 0.6-0.8 times the dosage needed to produce a corresponding effect in a younger patient. In a preferred embodiment of this invention, the patient is at least 65 years of age. For example, a patient over 65 years of age may require a dosage that is 25% less than a younger person of the same weight, i.e., the dosage would be adjusted by multiplying the weight-based dosage by a factor of 0.75.

### EXAMPLES

Patients undergoing elective colonoscopy were intravenously administered a 35 mg/mL sterile aqueous solution of O-phosphonooxyrnethyl propofol disodium salt for sedation. The patients refrained from ingesting caffeinated drinks and foods or alcohol for at least 12 hours prior to the administration of the prodrug. Opioids were discontinued 72 hours prior, and benzodiazepines and barbiturates were discontinued within 14 days of the start, with the exception of phenobarbital, which was discontinued 21 days prior. One i.v. catheter was placed in each arm, with one catheter designated for drug administration only and the other to draw blood samples.

The solutions were stored in a secure refrigerator (2° to 8° C.) until ready for use. The solutions were allowed to reach room temperature (approximately 1 hour) and kept at room temperature for up to 24 hours prior to administration. Fentanyl was used for pretreatment to mitigate paresthesias. Fentanyl, equivalent to 50 μg (0.05 mg) fentanyl base per mL, packaged in 2-mL and 5-mL DOSETTE® ampules (commercially available), was administered as an initial i.v. bolus dose of 1.5 μg/kg.

Some of the patients were randomized and others were assigned to a cohort as more dosing information became available. The intent was to enter 10 patients who were <60 years of age into each of the 10 mg/kg and 12.5 mg/kg propofol prodrug doses to determine the dose response to varying fentanyl and propofol prodrug doses. Likewise, for patients >60 years of age, the intent was to explore the 7.5 mg/kg and 10 mg/kg propofol prodrug doses with varying pretreatment doses of fentanyl.

The Modified Observer's Assessment of Alertness/Sedation (MOAA/S) scale was used to clinically rate the level of sedation. This evaluation places a grading score of 0 (does not respond to painful stimulus) to 5 (alert) in the category of responsiveness, as detailed in Table 1. An assessment of the Modified OAA/S score was made just prior to the fentanyl pretreatment and recorded at 1-minute intervals until the colonoscopy procedure began, then at 2-minute intervals throughout the procedure, and then at 1- or 3-minute intervals until the patient was fully alert.

TABLE 1
Responsiveness Scores of the Modified OAA/S Scale

| Responsiveness | Score |
|---|---|
| Responds readily to name spoken in normal tone | 5 (Alert) |
| Lethargic response to name spoken in normal tone | 4 |
| Responds only after name is called loudly and/or repeatedly | 3 |
| Responds only after mild prodding or shaking | 2 |
| Responds only after painful trapezius squeeze | 1 |
| Does not respond to painful trapezius squeeze | 0 |

Up to 4 supplemental doses of the propofol prodrug (1.5 to 5.0 mg/kg each) were permitted to achieve adequate sedation levels at the prescribed dose prior to the beginning of the colonoscopy, and to maintain adequate sedation levels throughout the procedure. In the event that insufficient analgesia is present (e.g., as evidenced by increased heart rate and blood pressure in the presence of adequate sedation), supplemental doses of fentanyl could be administered as needed. Supplemental oxygen was administered only if the oxygen saturation (SPO₂ as determined by pulse oximetry) fell below 90%, or if medical intervention was required. Supplemental oxygen (2 L/min nasally) was administered to all patients, beginning just prior to dosing and continued until the patient was deemed fully recovered.

### Example 1A

In this example, 93 patients were administered fentanyl and bolus propofol prodrug doses as summarized in Table 2 below.

TABLE 2
Propofol Prodrug Bolus Dose Group

| | | 7.5 mg/kg | 10 mg/kg | 12.5 mg/kg | Total |
|---|---|---|---|---|---|
| Fentanyl | 0.5 μg/kg | 4 | 13 | 10 | 27 |
| Dosing | 1.0 μg/kg | 9 | 14 | 11 | 34 |
| | 1.5 μg/kg | 11* | 10 | 11 | 32 |
| | Total | 24 | 37 | 32 | 93 |

*One patient received 1.5 μg/kg fentanyl + 8.5 μg/kg bolus dose of propofol prodrug

Demographic data for patients in Example 1A are summarized by original assignment to the treatment group (mg/kg) in Table 3.

TABLE 3
Demographic and Other Baseline Characteristics by Treatment Group
(Example 1A)

| | Bolus Dose Group | | |
|---|---|---|---|
| | 7.5 mg/kg | 10.0 mg/kg | 12.5 mg/kg |
| | (n = 24) | (n = 37) | (n = 32) |
| Age (years) | | | |
| Mean | 53.8 | 49.1 | 49.3 |
| Median | 59.0 | 51.0 | 51.5 |
| SD | 17.2 | 11.7 | 9.7 |
| Range | 24.0-80.0 | 20.0-73.0 | 20.0-60.0 |
| Gender (%) | | | |
| Male | 10 (41.7) | 19 (51.4) | 16 (50.0) |
| Female | 14 (58.3) | 18 (48.7) | 16 (50.0) |
| Race (%) | | | |
| Asian | 1 (4.2) | 0 (0.0) | 0 (0.0) |
| Black | 2 (8.3) | 4 (10.8) | 4 (12.5) |
| Caucasian | 16 (66.7) | 29 (78.4) | 24 (75.0) |
| Hispanic | 5 (20.8) | 4 (10.8) | 4 (12.5) |
| Other | 0 (0.0) | 0 (0.0) | 0 (0.0) |
| Weight (kg) | | | |
| Mean | 81.5 | 79.2 | 85.4 |
| Median | 81.0 | 79.4 | 81.5 |
| SD | 20.7 | 16.1 | 23.5 |
| Range | 51.2-129.7 | 51.9-133.8 | 51.2-135.1 |
| BMI | | | |
| Mean | 28.7 | 27.1 | 28.8 |
| Median | 26.0 | 27.0 | 28.5 |
| SD | 6.5 | 5.1 | 6.6 |
| Range | 21.0-45.0 | 19.0-49.0 | 20.0-43.0 |

N = Number;
SD = Standard deviation.

In general, demographic and baseline characteristics were similar across the 3 treatment groups, except for age. The patients in the 7.5 mg/kg bolus dose group were older (median age, 59 years) compared with the 10.0 and 12.5 mg/kg groups (51.0 and 51.5 years, respectively).

In general, results were similar for demographic data summarized by quartile of initial bolus dose (mg), as summarized in Table 4:

TABLE 4
Demographic and Other Baseline Characteristics by Quartile of Initial
Bolus Dose in mg (Example 1A)

| | Initial Bolus Propofol Prodrug Dose (mg) | | | | |
|---|---|---|---|---|---|
| | | 620 to | 777 to | | All |
| | <620 | <777 | <955 | ≧955 | Patients |
| | (n = 23) | (n = 23) | (n = 23) | (n = 24) | (N = 93) |
| Age (years) | | | | | |
| Mean | 52.0 | 47.7 | 51.0 | 50.8 | 50.4 |
| Median | 56.0 | 47.0 | 51.0 | 52.5 | 52.0 |
| SD | 18.2 | 10.8 | 11.5 | 9.5 | 12.8 |
| Range | 20.0-80.0 | 23.0-62.0 | 25.0-73.0 | 20.0-68.0 | 20.0-80.0 |
| Gender (%) | | | | | |
| Male | 8 (34.8) | 7 (30.4) | 13 (56.5) | 17 (70.8) | 45 (48.4) |
| Female | 15 (65.2) | 16 (69.6) | 10 (43.5) | 7 (29.2) | 48 (51.6) |
| Race (%) | | | | | |
| Asian | 1 (4.4) | 0 (0.0) | 0 (0.0) | 0 (0.0) | 1 (1.1) |
| Black | 1 (4.4) | 2 (8.7) | 3 (13.0) | 4 (16.7) | 10 (10.8) |
| Caucasian | 18 (78.3) | 16 (69.6) | 19 (82.6) | 16 (66.7) | 69 (74.2) |
| Hispanic | 3 (13.0) | 5 (21.7) | 1 (4.4) | 4 (16.7) | 13 (14.0) |
| Other | 0 (0.0) | 0 (0.0) | 0 (0.0) | 0 (0.0) | 0 (0.0) |
| Weight (kg) | | | | | |
| Mean | 66.5 | 72.8 | 85.8 | 101.8 | 81.9 |
| Median | 65.1 | 73.3 | 83.0 | 102.5 | 80.7 |
| SD | 10.7 | 13.3 | 17.3 | 17.1 | 20.1 |
| Range | 51.2-83.9 | 51.2-105.3 | 63.9-129.7 | 78.9-135.1 | 51.2-135.1 |
| BMI | | | | | |
| Mean | 24.2 | 25.7 | 28.9 | 33.3 | 28.1 |
| Median | 24.0 | 25.0 | 27.0 | 32.0 | 27.0 |
| SD | 3.5 | 4.6 | 5.7 | 5.7 | 6.0 |
| Range | 19.0-34.0 | 20.0-37.5 | 22.0-45.0 | 24.0-49.0 | 19.0-49.0 |

N = Number;
SD = Standard deviation.

Overall, 51.6% of patients were female, and 74.2% of patients were Caucasian. The patients in the lowest quartile (<620 mg) were older compared with patients in the other 3 quartiles. The median weight and body mass index (BMI) of patients increased with increasing quartiles of initial bolus doses, as would be expected by the quartile definitions. Of the 93 treated patients, two patients received alternative (rescue) medication and were considered treatment failures.

### Example 1B

In this example, the initial dosing scheme was divided into 2 weight groups, each with fixed doses of fentanyl and the propofol prodrug. Some of the initial patients weighed between 75 and 80 kg and were dosed with 980 mg (28 mL). During the initial portion, these subjects became more heavily sedated (MOAA/S<2) and experienced mild hypoxemia (oxygen saturation <90%) than was anticipated or desired. As a result, the lower boundary for the highest weight range was changed from >75 to >80 kg. Table 5 summarizes the adjusted weight-based, fixed-dosing schedules.

TABLE 5
Weight-Based, Fixed-Dosing Schedule

| | | | |
|---|---|---|---|
| Patient's | Pretreatment | Initial Propofol | Propofol prodrug |
| Weight | Fentanyl Dose | prodrug Bolus Dose | Supplemental Dose |

18 to 70 Years of Age

| | | | |
|---|---|---|---|
| ≦50 kg | 50 μg | 630 mg (18 mL) | up to 105 mg (3 mL) |
| >50 kg to | 60 μg | 700 mg (20 mL) | up to 140 mg (4 mL) |
| ≦65 kg | | | |
| >65 kg to | 70 μg | 805 mg (23 mL) | up to 175 mg (5 mL) |
| ≦80 kg | | | |
| >80 kg | 80 μg | 910 mg (26 mL) | up to 175 mg (5 mL) |

Patients ≧ 70 to 85 Years of Age and/or
with Hepatic or Renal Impairment

| | | | |
|---|---|---|---|
| ≦75 kg | 30 μg | 630 mg (18 mL) | up to 105 mg (3 mL) |
| >75 kg | 50 μg | 700 mg (20 mL) | |

Demographic data for patients in Example 1B are summarized by initial dose group (mg) in Table 6.

TABLE 6
Demographic and Other Baseline Characteristics by Initial Dose
(Example 1B)

| | Bolus Dose Group | | | | |
|---|---|---|---|---|---|
| | 630/700 mg | 805 mg | 910 mg | 980 mg | All |
| | (n = 9) | (n = 16) | (n = 18) | (n = 21) | Patients (N = 64) |
| Age (years) | | | | | |
| Mean | 63.8 | 44.9 | 52.8 | 50.5 | 51.6 |
| Median | 71.0 | 47.0 | 55.5 | 53.0 | 52.5 |
| SD | 19.5 | 11.6 | 12.0 | 11.3 | 13.9 |
| Range | 23.0-85.0 | 30.0-65.0 | 26.0-71.0 | 25.0-66.0 | 23.0-85.0 |
| Gender (%) | | | | | |
| Male | 2 (22.2) | 3 (18.8) | 10 (55.6) | 11 (52.4) | 26 (40.6) |
| Female | 7 (77.8) | 13 (81.3) | 8 (44.4) | 10 (47.6) | 38 (59.4) |
| Race (%) | | | | | |
| Asian | 1 (11.1) | 1 (6.3) | 0 (0.0) | 0 (0.0) | 2 (3.1) |
| Black | 0 (0.0) | 2 (12.5) | 2 (11.1) | 1 (4.8) | 5 (7.8) |
| Caucasian | 8 (89.0) | 12 (75.0) | 15 (83.3) | 19 (90.5) | 54 (84.4) |
| Hispanic | 0 (0.0) | 1 (6.3) | 1 (5.6) | 0 (0.0) | 2 (3.1) |
| Other | 0 (0.0) | 0 (0.0) | 0 (0.0) | 1 (4.8) | 1 (1.6) |
| Weight (kg) | | | | | |
| Mean | 66.9 | 64.1 | 105.4 | 100.5 | 88.0 |
| Median | 58.5 | 61.5 | 102.4 | 99.6 | 89.6 |
| SD | 21.5 | 10.1 | 18.1 | 15.1 | 24.3 |
| Range | 45.4-105.0 | 52.6-87.5 | 81.4-142.4 | 76.7-125.6 | 45.4-142.4 |
| BMI (kg/m²) | | | | | |
| Mean | 24.2 | 24.0 | 35.9 | 32.8 | 30.3 |
| Median | 24.0 | 23.0 | 35.0 | 33.0 | 30.0 |
| SD | 5.5 | 3.5 | 6.2 | 4.7 | 7.1 |
| Range | 17.0-35.0 | 19.0-33.0 | 27.0-54.0 | 24.0-44.0 | 17.0-54.0 |

N = Number;
SD = Standard deviation.

The median age of patients in the 630/700 mg dose group was higher (71.0 years) compared with patients in the 805-, 910-, and 980-mg groups (range, 47.0 to 55.5 years). Overall, 59.4% of patients were female and 84.4% of patients were Caucasian. Patients in the 630/700-mg and 805-mg groups had more females than males and lower median weight and BMI compared with the higher dose groups (910 and 980 mg). Of the 64 treated patients, two patients received alternative (rescue) medication and were considered treatment failures.

### Results

Efficacy data collected in both Examples 1A and 1B were summarized using descriptive statistics. The Modified OAA/S results were summarized as mean, median, and frequency distribution for each time point and grouped by each dose level. For each patient, the time interval when the Modified OAA/S score was ≧2 and ≦4 was calculated using a midpoint approach. Data for individual Modified OAA/S scores were summarized in minute (±30 seconds) intervals and displayed in figures for the following 3 time intervals: prior to procedure is displayed by 1-minute intervals; during the procedure is displayed by 2-minute intervals; and recovery period is displayed by 3-minute intervals. The last observation carried forward (LOCF) was used when the observation in the designated time window was missing.

The percent of time that the Modified OAA/S score was ≧2 and ≦4 during the colonoscopy procedure was calculated as the sum of all intervals with Modified OAA/S scores ≧2 and ≦4 divided by duration of the procedure ×100% for each patient, and summarized by dose group.

Tables 7 and 8 summarize the time required, in minutes, from the initial bolus administration to achieve sedation by quartile of initial bolus dose (mg) and by total dose (mg), respectively, in Example 1A.

TABLE 7
Time (Minutes) from Propofol Prodrug Dose to Achieve Sedation by
Quartile of Initial Bolus Dose in mg (Example 1A)

| Time | | | | | |
|---|---|---|---|---|---|
| (minutes) | Initial Bolus Dose (mg) | | | | All |
| to Achieve | <620 | 620 to <777 | 777 to <955 | ≧955 | Patients |
| Sedation* | (n = 24) | (n = 23) | (n = 23) | (n = 23) | (N = 91) |
| Mean | 4.3 | 2.7 | 2.0 | 1.8 | 2.7 |
| Median | 3.0 | 2.5 | 2.0 | 2.0 | 2.0 |
| SD | 4.4 | 1.6 | 1.6 | 1.1 | 2.6 |
| Range† | −5.3 to | −1.0 to 6.0 | −0.8 to 7.0 | −2.0 to | −5.3 to |
| | 14.0 | | | 3.0 | 14.0 |

SD = Standard deviation.
*Time (minutes) interval from first propofol prodrug administration to first Modified OAA/S score ≦4.
†Negative values represent patients who achieved sedation following fentanyl and prior to propofol prodrug.

TABLE 8
Time (Minutes) from Propofol Prodrug Dose to Achieve Sedation by
Quartile of Total Dose in mg (Example 1A)

| Time (minutes) to | Total Propofol Prodrug Dose (mg) | | | |
|---|---|---|---|---|
| Achieve | <762 | 762 to <891 | 891 to <1041 | ≧1041 |
| Sedation* | (n = 24) | (n = 23) | (n = 23) | (n = 23) |
| Mean | 2.3 | 2.3 | 3.8 | 2.3 |
| Median | 2.0 | 2.0 | 2.6 | 2.0 |
| SD | 2.6 | 1.0 | 4.3 | 1.2 |
| Range† | −5.3 to 7.0 | 1.0 to 5.0 | −2.0 to 14.0 | 1.0 to 6.0 |

SD = Standard deviation.
*Time (minutes) interval from first propofol prodrug administration to first Modified OAA/S score ≦4.
†Negative values represent patients who achieved sedation following fentanyl and prior to propofol prodrug.

For all patients in Example 1A, the median time from initial bolus administration to achieve sedation (first Modified OAA/S score ≦4) was 2.0 minutes. The median time to achieve sedation decreased with increasing initial bolus doses; however, no dose-response was observed when the data was analyzed as total dose.

Because fentanyl was given approximately 5 minutes prior to the administration of the initial bolus dose according to the protocol, the time from fentanyl administration to achieve sedation and to start of colonoscopy was approximately 5 minutes longer than the time calculated from the initial bolus dose of propofol prodrug.

Table 9 summarizes the time from the administration of the propofol prodrug to achieve sedation and the time to the start of the colonoscopy procedure for Example 1B.

TABLE 9
Time from Propofol Prodrug Dose to Achieve Sedation and to Procedure
Start by Initial Bolus Dose (Example 1B)

| Initial Bolus Propofol Prodrug Dose | | | | All |
|---|---|---|---|---|
| 630/700 mg | 805 mg | | | Patients |
| (n = 8) | (n = 16) | 910 mg (n = 17) | 980 mg (n = 21) | (N = 62) |

Time (minutes) to Achieve Sedation*

| | | | | | |
|---|---|---|---|---|---|
| Mean | 1.8 | 2.5 | 3.7 | 2.4 | 2.7 |
| Median | 2.0 | 2.0 | 3.0 | 2.0 | 2.0 |
| SD | 2.4 | 2.1 | 3.1 | 0.7 | 2.2 |
| Range† | −3.0 to 5.0 | −3.0 to 7.0 | −1.0 to 10.0 | 1.0 to 4.0 | −3.0 to 10.0 |

Time (minutes) to Procedure Start‡

| | | | | | |
|---|---|---|---|---|---|
| Mean | 3.6 | 3.6 | 5.2 | 2.9 | 3.8 |
| Median | 3.5 | 3.0 | 4.0 | 3.0 | 3.0 |
| SD | 1.1 | 2.7 | 2.6 | 0.9 | 2.2 |
| Range† | 2.0 to 5.0 | 2.0 to 13.0 | 2.0 to 11.0 | 1.0 to 4.0 | 1.0 to 13.0 |

SD = Standard deviation.
*Time (minutes) interval from first propofol prodrug administration to first Modified OAA/S score ≦4.
†Negative values represent patients who achieved sedation following fentanyl and prior to administration of the propofol prodrug.
‡Time (minutes) interval from first propofol prodrug administration to start of the colonoscopy procedure.

For all patients, the median time from initial bolus administration to achieve sedation (defined as first Modified OAA/S score ≦4) was 2.0 minutes. Most patients achieved sedation within 2 to 3 minutes. The median time from the initial bolus administration to procedure start was 3.0 minutes. No dose-related trend was noted in time to procedure start.

Table 10A summarizes the time (minutes) from withdrawal of the colonoscope at the end of the procedure until the patient met the criteria for fully alert, fully recovered, and ready for discharge, the dose-ranging portion of the study by quartile of initial bolus dose (mg).

TABLE 10A
Time (Minutes) to Fully Alert, Fully Recovered, and Ready for Discharge
by Quartile of Initial Bolus Dose in mg (Example 1A)

| Initial Bolus Propofol Prodrug Dose (mg) | | | | |
|---|---|---|---|---|
| <620 | 620 to <777 | 777 to <955 | ≧955 | All Patients |
| (n = 24) | (n = 23) | (n = 23) | (n = 23) | (N = 91) |

Time (minutes) to Fully Alert

| | | | | | |
|---|---|---|---|---|---|
| N | 22 | 23 | 23 | 23 | 91 |
| Mean | 11.5 | 9.3 | 12.0 | 12.0 | 11.2 |
| Median | 12.5 | 8.0 | 13.0 | 11.0 | 11.0 |
| SD | 5.2 | 5.3 | 5.1 | 7.3 | 5.8 |
| Range | 1-20 | 1-20 | 1-19 | 0-28 | 0-28 |

Time (minutes) to Fully Recovered

| | | | | | |
|---|---|---|---|---|---|
| N | 20 | 23 | 23 | 22 | 88‡ |
| Mean | 18.4 | 17.1 | 21.7 | 22.9 | 20.1 |
| Median | 20.5 | 17.0 | 21.0 | 20.5 | 20.0 |
| SD | 7.7 | 7.7 | 9.5 | 10.0 | 9.0 |
| Range | 4-40 | 5-33 | 4-51 | 5-49 | 4-51 |

Time (minutes) to Ready for Discharge

| | | | | | |
|---|---|---|---|---|---|
| N | 22 | 23 | 23 | 23 | 91 |
| Mean | 32.9 | 35.8 | 39.6 | 40.2 | 37.2 |
| Median | 33.0 | 34.0 | 38.0 | 40.0 | 37.0 |
| SD | 10.7 | 15.2 | 12.5 | 8.9 | 12.2 |
| Range | 12-57 | 15-89 | 22-64 | 27-63 | 12-89 |

N = Number;
SD = Standard deviation.
‡Data were not collected for 3 patients.

Overall, the median times from withdrawal of the colonoscope until patients met the criteria for fully alert, fully recovered, and ready for discharge were 11.0, 20.0, and 37.0 minutes, respectively. The median time to ready for discharge increased with increasing initial bolus doses; however, the median time to fully alert and fully recovered were lowest (8.0 and 17.0 minutes, respectively) in the second quartile of initial bolus doses (620 mg to <777 mg).

Table 10AA summarizes the time (minutes) from withdrawal of the colonoscope at the end of the procedure until the patient met the criteria for fully alert, fully recovered, and ready for discharge by quartile of total dose (mg) in the patients in Part 1A.

TABLE 10AA
Time (Minutes) to Fully Alert, Fully Recovered, and Ready for
Discharge by Quartile of Total Dose in mg (Example 1A)

Total Propofol Prodrug Dose (mg)

| | | | |
|---|---|---|---|
| | | 891 to | |
| <762 | 762 to <891 | <1041 | ≧1041 |
| (n = 24) | (n = 23) | (n = 23) | (n = 23) |

Time (minutes) to Fully Alert

| | | | | |
|---|---|---|---|---|
| N | 22 | 23 | 23 | 23 |
| Mean | 9.2 | 11.3 | 11.6 | 12.6 |
| Median | 8.0 | 11.0 | 11.0 | 12.0 |
| SD | 4.9 | 4.6 | 5.9 | 7.3 |
| Range | 1-17 | 4-20 | 1-20 | 0-28 |

Time (minutes) to Fully Recovered

| | | | | |
|---|---|---|---|---|
| N | 21 | 23 | 22 | 22 |
| Mean | 19.0 | 19.0 | 20.5 | 21.8 |
| Median | 21.0 | 18.0 | 19.5 | 20.5 |
| SD | 7.8 | 9.5 | 8.0 | 10.5 |
| Range | 4-40 | 4-51 | 5-33 | 5-49 |

Time (minutes) to Ready for Discharge

| | | | | |
|---|---|---|---|---|
| N | 22 | 23 | 23 | 23 |
| Mean | 33.8 | 36.3 | 38.5 | 39.9 |
| Median | 35.0 | 32.0 | 38.0 | 40.0 |
| SD | 9.5 | 12.9 | 14.7 | 11.1 |
| Range | 12-57 | 18-64 | 17-89 | 15-63 |

N = Number;
SD = Standard deviation.

The median time to fully alert was lowest (8.0 minutes) in the lowest quartile (<762 mg of total dose administered) and increased with the higher doses, whereas the median times to fully recovered and ready for discharge were lowest (18 and 32 minutes, respectively) in the second quartile (762 mg to <891 mg).

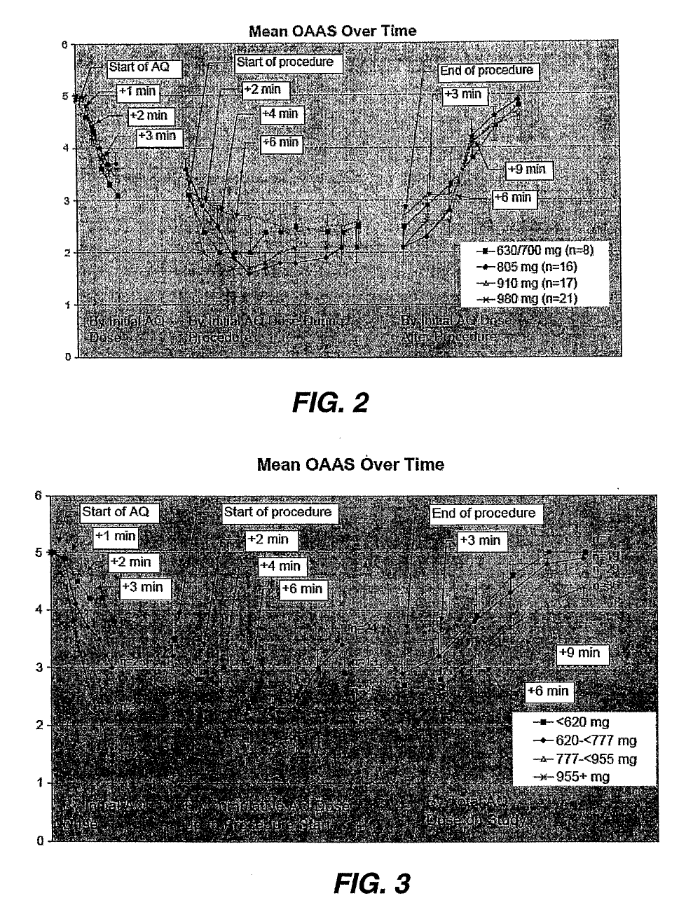

FIG. 2 shows the mean Modified OAA/S over time at each procedural period by initial bolus dose for Example 1A. The figure is divided into 3 analyses: Modified OAA/S score by quartile of initial bolus dose; Modified OAA/S score by quartile of cumulative dose prior to the procedure; and Modified OAA/S scores by quartile of cumulative total dose during recovery.

For the initial dose, mild to moderate sedation was achieved earlier as the initial bolus dose of was increased by quartiles. Induction of mild to moderate sedation levels (Modified OAA/S≧2 and ≦4) was achieved within 2 minutes following administration of 620 mg to 955 mg of propofol prodrug. Only the highest dosing quartile (955 mg and above) reached a mean Modified OAA/S of <2, and did so between 4 and 5 minutes.

Similarly, as the cumulative doses were analyzed by the original quartile doses during the procedure, the higher dose quartiles demonstrated deeper sedation scores which lasted longer. Maintenance of mild to moderate sedation levels (Modified OAA/S≧2 and ≦4) during the procedure (12-30 minutes) was achieved following administration of 620 mg to 955 mg. The mean Modified OAA/S scores only fell below 2 during the procedure when cumulative doses were >777 mg. Importantly, a mean Modified OAA/S score ≧2 and ≦4 was maintained during the colonoscopy with cumulative doses from 620 mg to 955 mg.

Recovery from mild to moderate sedation to a Modified OAA/S of 5 occurred within 9 to 12 minutes after end of procedure at cumulative doses from 620 mg to 955 mg. Mean Modified OAA/S scores returned to 5 sooner with decreasing total doses.

Table 10B summarizes the time from withdrawal of the colonoscope at the end of the procedure until the patients met the criteria for fully alert, fully recovered, and ready for discharge by initial bolus dose for Example 1B.

TABLE 10B
Time to Fully Alert, Fully Recovered, and Ready for Discharge by Initial
Bolus Dose (Example 1B)

| Initial Bolus Propofol Prodrug Dose | | | | |
|---|---|---|---|---|
| 630/700 mg | 805 mg | | | All Patients |
| (n = 8) | (n = 16) | 910 mg (n = 17) | 980 mg (n = 21) | (N = 62) |

Time (minutes) to Fully Alert

| | | | | | |
|---|---|---|---|---|---|
| Mean | 12.5 | 12.2 | 11.5 | 13.7 | 12.5 |
| Median | 12.5 | 12.5 | 11.0 | 12.0 | 12.0 |
| SD | 6.1 | 4.8 | 6.9 | 8.0 | 6.7 |
| Range | 5.0 to 22.0 | 1.0 to 19.0 | 3.0 to 26.0 | 0.0 to 31.0 | 0.0 to 31.0 |

Time (minutes) to Fully Recovered

| | | | | | |
|---|---|---|---|---|---|
| Mean | 20.0 | 20.3 | 25.6 | 21.5 | 22.1 |
| Median | 19.0 | 18.0 | 21.0 | 20.0 | 20.0 |
| SD | 7.1 | 12.8 | 26.5 | 11.0 | 16.6 |
| Range | 10.0 to 30.0 | 10.0 to 66.0 | 3.0 to 122.0 | 2.0 to 47.0 | 2.0 to 122.0 |

Time (minutes) to Ready for Discharge

| | | | | | |
|---|---|---|---|---|---|
| Mean | 39.4 | 41.8 | 40.4 | 39.5 | 40.3 |
| Median | 36.5 | 35.0 | 33.0 | 37.0 | 35.5 |
| SD | 13.4 | 16.4 | 28.2 | 10.3 | 18.2 |
| Range | 28.0 to 68.0 | 20.0 to 69.0 | 12.0 to 138.0 | 26.0 to 62.0 | 12.0 to 138.0 |

SD = Standard deviation.

Overall, the median times from withdrawal of the colonoscope until patients met the criteria for fully alert, fully recovered, and ready for discharge were 12.0, 20.0, and 35.5 minutes, respectively. Results were similar for the 630/700 mg, 805 mg, and 980-mg dose groups for median time to fully alert (range, 12.0 to 12.5 minutes), fully recovered (range, 18.0 to 20.0 minutes), and ready for discharge (range, 35.0 to 37.0 minutes). The 910 mg dose group had a shorter median time to fully alert (11.0 minutes) and ready for discharge (33.0 minutes), but a longer time to fully recovered (21.0 minutes) than the other 3 dose groups.

FIG. 3 shows the mean Modified OAA/S score over time at each procedural period by initial bolus dose of the propofol prodrug for Example 1B. Following the start of procedure, the mean Modified OAA/S scores fell below 2 within 4 minutes with an initial bolus dose of 980 and within 6 minutes with an initial bolus dose of 805 mg, and remained <2 throughout most of the procedure for both doses.

A mean Modified OAA/S score ≧2 and ≦4 was maintained during the majority of the colonoscopy with initial bolus doses of 630/700 mg or 910 mg. The mean Modified OAA/S scores ranged below 2 in the 805 mg and the 980 mg groups. At the end of the colonoscopy, mean Modified OAA/S scores returned to 5 (alert) earlier with an initial bolus dose of 805 mg.

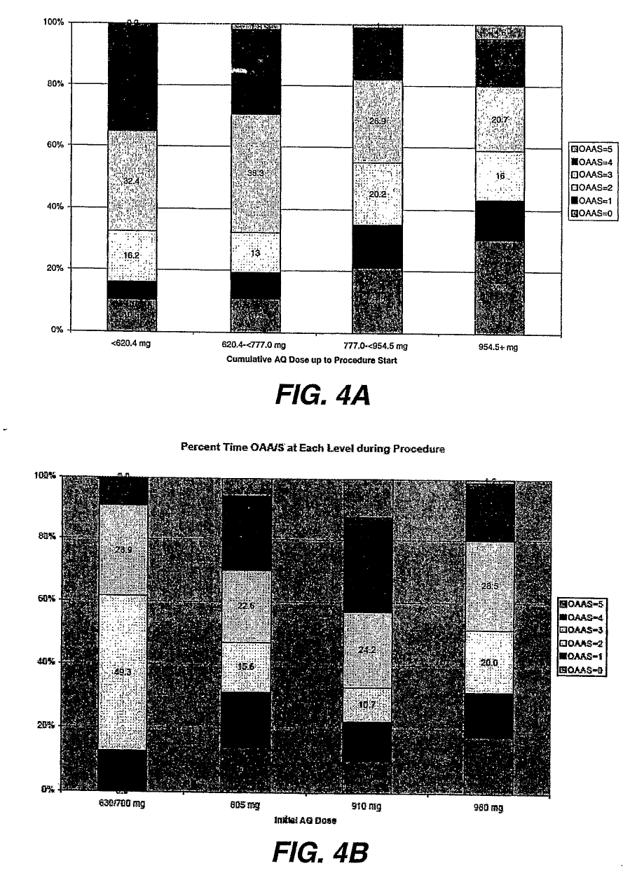

FIG. 4A shows the percent of time patients were at each OAA/S sedation level during colonoscopy for Example 1A, quartiled by cumulative dose up to the procedure start. The percent of time that patients remained within Modified OAA/S levels of sedation of 2, 3, or 4 was highest among patients who received <620 mg up to procedure start (83.6%) and decreased with increasing initial bolus doses of (range, 78.9% to 52.2%). The percent of procedural time at MOAA/S levels that are more consistent with deep sedation increased as the bolus dose increased (range, 16.2% to 43.2%).

FIG. 4B illustrates the percent of time that the patients were at each Modified OAA/S score during colonoscopy by initial bolus dose. The percent of time that the patients remained within Modified OAA/S scores of 2, 3, or 4 from procedure start to procedure end was highest among patients who received 630/700 mg doses (87.5%) compared with initial bolus doses of 805, 910, and 980 mg (range, 61.9% to 67.3%). The percent of time spent at Modified OAA/S scores of 1 or 0 was highest with the initial bolus doses of 805 and 980 mg (31.0% and 31.5%, respectively) compared with 630/700 mg and 910 mg (12.5% and 22.3%, respectively).

Table 11A summarizes the number of doses by quartile of initial bolus dose (mg) needed to allow the start of the colonoscopy procedure in Example 1A.

TABLE 11A
Number of Doses Required to Allow Start of Procedure by Quartile of
Initial Bolus Dose in mg (Example 1A)

| | Initial Bolus Dose (mg) | | | | |
|---|---|---|---|---|---|
| | <620 | 620 to <777 | 777 to <955 | ≧955 | All Patients |
| Number | (n = 24) | (n = 23) | (n = 23) | (n = 23) | (N = 91) |
| of Doses | Number of Patients | | | | |
| 1 | 10 | 20 | 21 | 22 | 73 |
| 2 | 7 | 1 | 2 | 1 | 11 |
| 3 | 4 | 2 | 0 | 0 | 6 |
| 4 | 1 | 0 | 0 | 0 | 1 |

For 73 (80.2%) patients, the initial bolus dose was sufficient to start the procedure. The number of patients who needed supplemental doses to allow the start of procedure decreased with increasing initial bolus dose: 12 (54.5%), 3 (13.0%), 2 (8.7%), and 1 (4.3%), for dosing groups <620 mg, 620-<777 mg, 777-<955 and ≧955, respectively.

Table 11B summarizes the number of doses, by initial bolus dose (mg), that were needed to allow the start of the colonoscopy procedure for Example 1B.

TABLE 11B
Number of Doses Required to Allow Start of Procedure (Example 1B)

| | Initial Bolus Propofol Prodrug Dose (mg) | | | | |
|---|---|---|---|---|---|
| | 630/700 mg | 805 mg | 910 mg | 980 mg | All Patients |
| Number | (n = 8) | (n = 16) | (n = 17) | (n = 21) | (N = 62) |
| of Doses | Number of Patients | | | | |
| 1 | 8 | 14 | 13 | 21 | 56 |
| 2 | 0 | 2 | 4 | 0 | 6 |
| 3 | 0 | 0 | 0 | 0 | 0 |
| 4 | 0 | 0 | 0 | 0 | 0 |

For 56 (90.3%) patients, the initial bolus dose was sufficient to start the procedure. Two (12.5%) in 805-mg group and four (23.5%) in the 910-mg group required one supplemental dose to start the procedure.

Table 12A summarizes the number of doses by quartile of initial bolus dose (mg) required to maintain sedation throughout the colonoscopy procedure in Example 1A.

TABLE 12A
Number of Supplemental Doses Required to Maintain Sedation
Throughout the Procedure by Quartile of Initial Bolus Dose in mg
(Example 1A)

| | Initial Bolus Dose (mg) | | | | |
|---|---|---|---|---|---|
| Number of | <620 | <777 | 777 to <955 | ≧955 | All Patients |
| Supplemental | (n = 24) | (n = 23) | (n = 23) | (n = 23) | (N = 91) |
| Doses | Number of Patients | | | | |
| 0 | 7 | 11 | 16 | 21 | 55 |
| 1 | 10 | 11 | 5 | 2 | 28 |
| 2 | 5 | 1 | 2 | 0 | 8 |

SD = Standard deviation.

Most patients required no supplemental dosing, only 8 (8.8%) required more than 1 supplemental dose. The incidence of patients who required supplemental doses to maintain sedation decreased with increasing initial bolus doses: 15 (68.2%), 12 (52.2%), 7 (30.4%), and 2 (8.7%), <620 mg, 620-<777 mg, 777-<955 and ≧955, respectively.

Table 12B summarizes the number of doses by initial bolus dose (mg) required to maintain sedation throughout the colonoscopy procedure for Example 1B.

TABLE 12B
Number of Supplemental Doses Required to Maintain Sedation
Throughout the Procedure by Initial Bolus Dose (Example 1B)

| | Initial Bolus Dose (mg) | | | | |
|---|---|---|---|---|---|
| Number of | 630/700 mg | 805 mg | 910 mg | 980 mg | All Patients |
| Supplemental | (n = 8) | (n = 16) | (n = 17) | (n = 21) | (N = 62) |
| Doses | Number of Patients | | | | |
| 1 | 1 | 5 | 3 | 6 | 15 |
| 2 | 0 | 3 | 4 | 0 | 7 |
| 3 | 0 | 0 | 1 | 0 | 1 |
| 4 | 0 | 0 | 0 | 0 | 0 |

Most patients did not require supplemental dosing. Twenty-three (37.1%) patients required at least 1 supplemental dose to maintain sedation. Fifteen (24.2%) required 1 supplemental dose; seven (11.3%) required 2 supplemental doses; and one (1.6%) in the 910-mg group required 3 supplemental doses. No dose-related trends could be observed for the requirement of supplemental dosing.

Table 13A summarizes the total number of doses (initial plus supplemental) by quartile of initial bolus dose (mg) required during the entire procedure in Example 1A.

TABLE 13A
Total Number of Doses Administered by Quartile of Initial Bolus Dose
in mg (Example 1A)

| | Initial Bolus Dose (mg) | | | | |
|---|---|---|---|---|---|
| | <620 | 620 to <777 | 777 to <955 | ≧955 | All Patients |
| Number of | (n = 24) | (n = 23) | (n = 23) | (n = 23) | (N = 91) |
| Doses* | Number of Patients | | | | |
| 1 | 4 | 10 | 15 | 20 | 49 |
| 2 | 6 | 10 | 6 | 3 | 25 |
| 3 | 6 | 1 | 1 | 0 | 8 |
| 4 | 4 | 2 | 1 | 0 | 7 |
| 5 | 2 | 0 | 0 | 0 | 2 |

*Initial plus supplemental doses.

The percentage of patients who required only a single dose to initiate and maintain sedation increased with increasing initial bolus doses: 4 (18.2%), 10 (43.5%), 15 (65.2%), and 20 (87.0%), respectively.

Table 13B summarizes the total number of doses (initial plus supplemental) by initial bolus dose (mg) required during the entire procedure for Example 1B.

TABLE 13B
Total Number of Doses Administered by Initial Bolus Dose in mg
(Example 1B)

| | Initial Bolus Dose | | | | |
|---|---|---|---|---|---|
| | 630/700 mg | 805 mg | 910 mg | 980 mg | All Patients |
| | (n = 8) | (n = 16) | (n = 17) | (n = 21) | (N = 62) |
| | Number of Patients | | | | |
| Number of | | | | | |
| Doses* | | | | | |
| 1 | 7 | 8 | 8 | 15 | 38 |
| 2 | 1 | 4 | 3 | 6 | 14 |
| 3 | 0 | 3 | 3 | 0 | 6 |
| 4 | 0 | 1 | 3 | 0 | 4 |
| Total Dose | | | | | |
| (mg) | | | | | |
| N | 8 | 16 | 17 | 21 | 62 |
| Mean | 700.0 | 945.0 | 1093.2 | 1030.0 | 982.8 |
| Median | 700.0 | 875.0 | 1085.0 | 980.0 | 980.0 |
| SD | 37.4 | 171.5 | 206.1 | 81.0 | 189.0 |
| Range | 630.0 to | 805.0 to | 910.0 to | 980.0 to | 630.0 to |
| | 770.0 | 1330 | 1435 | 1155 | 1435 |

SD = Standard deviation
*Initial plus supplemental doses

Overall, 52 (83.9%) of the 62 patients required ≦2 doses to initiate and maintain sedation to complete the procedure.

Venous blood plasma samples taken from the patients of Examples 1A and B at several points during and after the procedure were analyzed for concentrations of propofol prodrug and propofol derived from the propofol prodrug, and the obtained values (see FIG. 1) were added to a data pool for the purpose of developing a population pharmacokinetic model for predicting the concentrations of both compounds in venous plasma, and a pharmacokinetic/pharmacodynamic model for the level of sedation (MOAA/S score). In developing these models, the following parameters were included as covariates: gender, height, weight, age, race, body surface, lean body weight, body mass index, clinical laboratory values (albumin, aspartate, transaminase, bilirubin, serum creatinine, alkaline phosphatase, alanine transaminase), fentanyl (initial and total dose, weight-normalized initial and total dose, plasma fentanyl concentrations at 6 and 14 min. following administration) and propofol prodrug.

For predicting the concentrations of propofol and the prodrug in venous plasma, a linear 5-compartment model was applied using commercially available software (NONMEM, Version V, Level 1.1, Globomax L.L.C., East Hanover, Md.). Predictive check simulations of 500 trials yielded good agreement of predicted values with the observed data, with only slight under-estimation of propofol and prodrug concentrations, and slight over-estimation of prodrug variability. The results of this pharmacokinetic model showed an effect of lean body weight (LBW) on predicted plasma concentrations. Specifically, the central volumes of the prodrug, of propofol generated from the prodrug, and of clearance of the prodrug were increased by 1.8%, 2.5%, and 1.4% per kg of LBW over 55 kg, respectively. Gender and weight were strongly correlated, but there was no independent gender effect. Further, the model yielded no significant effects of fentanyl total dose or age.

It can be concluded that a linear pharmacokinetic model adequately describes the observed data from Examples 1A and B. Lean body weight was the best predictor of the concentrations of propofol generated from administration of the propofol prodrug. This finding has important implications for the dosing of overweight individuals for mild to moderate conscious sedation. Specifically, when the prodrug is dosed strictly proportional to body weight, obese patients are predicted to attain higher propofol plasma concentrations, and a deeper level of sedation, than may be necessary or desirable for their specific medical needs.

For predicting the level of sedation (MOAA/S score) attained after intravenous administration of the prodrug, two models were applied using commercially available software (NONMEM, Version V, Level 1.1, Globomax L.L.C., East Hanover, Md.). First, a probabilistic (proportional odds) model tested whether the logit function of the probability of the MOAA/S score to reach a certain level (0, 1, . . . 5) is a linear function of the concentration of propofol (generated from the prodrug) in the effect compartment. Second, a continuous population model tested whether the expected MOAA/S score is a Hill function of the effect-site concentration of propofol generated from the prodrug.

The probabilistic and continuous models were found to adequately describe the observed data and covariate effects with generally similar results. At the doses used in Examples 1A and B, the effect of fentanyl on sedation was small, and the models were not able to distinguish a fentanyl effect from the effect of propofol generated from the prodrug. The models detected no effect of gender. Importantly, both models predicted that subjects over the age of 65 are more sensitive to propofol generated after intravenous administration of the prodrug, relative to younger subjects. Specifically, the models were found to predict that, in subjects older than 65, a given MOAA/S score is attained at about 33% (probabilistic model) to about 25% (continuous model) lower effect site concentrations than in younger subjects. Taking into account an adequate margin of safety, a dose reduction of about 20% to about 40% is warranted for patients over 60 years, for whom adequate conscious sedation is desired during short surgical and diagnostic procedures.

While particular embodiments of the present invention have been described and illustrated, it should be understood that the invention is not limited thereto since modifications may be made by persons skilled in the art. The present application contemplates any and all modifications that fall within the spirit and scope of the underlying invention disclosed herein.

1. A method for determining a dose of a compound of Formula I effective for inducing mild to moderate sedation levels in a patient, 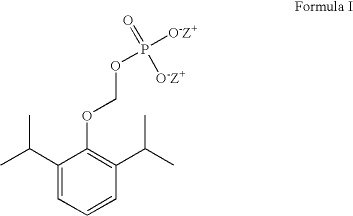
wherein each Z is independently selected from the group consisting of hydrogen, alkali metal ion, and amine, the method comprising determining a patient's lean body mass and calculating a dose based on lean body mass.

2. The method of claim 1 wherein the dose is effective for producing in the patient a Modified Observer's Assessment of Alertness/Sedation (MOAA/S) score of ≧2 and ≦4.

3. The method of claim 1 wherein the patient is overweight or obese.

4. The method of claim 3, wherein the dose is effective for inducing mild to moderate sedation after administration by one or more parenteral bolus injections.

5. The method of claim 3, wherein the dose for inducing mild to moderate sedation ranges from about 3 mg/kg lean body weight to about 30 mg/kg lean body weight.

6. The method of claim 5, wherein the dose ranges from about 3 mg/kg lean body weight to less than 23 mg/kg lean body weight.

7. The method of claim 6, wherein the dose ranges from about 7.5 mg/kg lean body weight to about 15 mg/kg lean body weight.

8. A method for inducing mild to moderate sedation in an overweight or obese patient, comprising: administering to said patient a compound of Formula I as described in claim 1 at a dose determined according to claim 1.

9. A method for inducing a pharmacological effect in an overweight or obese patient in need thereof, comprising: determining the patient's lean body weight, and administering to said patient a pharmacologically effective parenteral dose of a compound of Formula I as described in claim 1 in an amount proportional to said patient's lean body weight; wherein the pharmacological effect is selected from the group consisting of: treating an epileptic condition, treating nausea or vomiting; treating pruritus, treating migraine headache, inducing muscle relaxation, inducing unconsciousness, and inducing a conscious sedated state.

10. A method for determining a dose of a compound of Formula I suitable for inducing mild to moderate sedation levels in a patient who is at least 60 years of age, 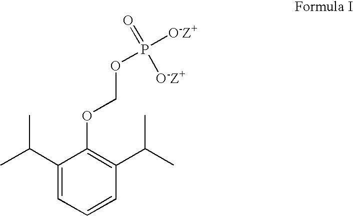
wherein each Z is independently selected from the group consisting of hydrogen, alkali metal ion, and amine, the method comprising determining a weight-appropriate dose for the patient and then adjusting the weight-appropriate dose by an age-based factor.

11. The method of claim 10 wherein the dose is determined by multiplying the weight-appropriate dose by an age-based factor of from about 0.6 to about 0.8.

12. The method of claim 11, wherein the patient is at least 65 years of age.

13. The method of claim 11 wherein the age-based factor is about 0.75.

14. The method of claim 10 wherein the dose is effective for producing in the patient a Modified Observer's Assessment of Alertness/Sedation (MOAA/S) score of ≧2 and ≦4.

15. The method of claim 11, wherein the dose is effective for inducing mild to moderate sedation after administration by one or more parenteral bolus injections.

16. A method for inducing mild to moderate sedation in a patient who is at least 60 years of age, comprising: administering to said patient a compound of Formula I as described in claim 10 at a dose determined according to claim 10.
